(19) United States
(12) Patent Application Publication
(10) Pub. No.: US 2024/0386479 A1
(43) Pub. Date: Nov. 21, 2024

(54) SYSTEM AND METHOD FOR RENTING MEDICAL DEVICES

(71) Applicant: Moshe Ein-Gal, Ramat Hasharon (IL)

(72) Inventor: Moshe Ein-Gal, Ramat Hasharon (IL)

(21) Appl. No.: 18/783,605

(22) Filed: Jul. 25, 2024

Related U.S. Application Data
(63) Continuation-in-part of application No. 17/382,451, filed on Jul. 22, 2021.

(51) Int. Cl. G06Q 30/0645; G16H 40/40

(52) U.S. Cl. CPC G06Q 30/0645; G16H 40/40

(57) ABSTRACT

A system for treatment includes an identification (ID) element, and an ID reader configured to read ID information provided by the ID element. The ID information includes an identifier of a user and authorization information for authorizing that user to use the medical device. The authorization information includes authorization that the user has paid a rental fee to use the medical device. A controller is in communication with the ID reader configured to process the ID information. A disabling element is capable of disabling operation of the medical device upon receiving a disabling signal from the controller if the controller cannot verify that the user is authorized to use the medical device.

### CROSS-REFERENCE TO OTHER APPLICATIONS

The present application is a continuation-in-part of, and claims priority from, U.S. patent application Ser. No. 17/382,451, filed 22 Jul. 2021.

### FIELD OF THE INVENTION

The present invention generally relates to renting or leasing of medical devices to improve medical care for the public without problems of home use.

### BACKGROUND OF THE INVENTION

Japanese Patent Application JP2002276177 describes a composite medical facility in which medical doctors can operate clinics in accordance with their medical treatment policies and management policies The composite medical facility has a plurality of clinics, which are respectively operated by different management subjects and, at the same time, can be leased, including various types of medical appliances installed in the clinics. In addition, the clinics are adjustable, that is, they can be changed in layout and the medical appliances which are used or leased can be changed to meet the demands of the lessor and lessees. The medical facility has an integrated receiving and accounting room, exclusively used inspection clinic, exclusively used medical treatment room, restaurant for outsiders, checkroom, and other commonly used facilities.

However, that patent application involves setting up many clinics, which are subject to medical and legal regulations, and the whole process and system becomes complicated and expensive.

In addition, medical devices for home use, such as wheelchairs, may be rented by users, i.e., care recipients. The rental period is typically long (e.g., weeks or months) and the device is used at the home of the care recipient. Multi-sessions and low risk treatments, such as treatment of erectile dysfunction by low intensity shockwaves, involve short sessions of less than one hour each. Due to the number of sessions and the time between successive sessions, the total overall treatment time is on the order of weeks or months. Electromedical devices for such multi-sessions treatments are more expensive than typical devices for home use, and rental of such devices for the duration of several weeks or months may be prohibitively expensive. Consequently, while such devices for multi-sessions treatments may be configured for home treatment, rental for such a long period is currently not implemented by care recipients. Thus, a need exists for a solution to the distribution problem of how to provide healthcare professionals with resources allowing the professional to perform medical treatments with minimum upfront investment and a need also exists for care recipients to afford renting devices for multi-sessions treatments.

### SUMMARY OF THE INVENTION

The present invention seeks to provide medical device rental outlets (as opposed to medical clinics) for renting electro-medical devices intended for multi-sessions treatments, wherein the rental period is short (for example, generally sufficient for conducting a session), and the session takes place at or close to the outlet. As opposed to medical clinics, such outlets are merely rental outlets, and as such, are subject to less regulations, are easier to manage, and are less expensive to set up and run. Consequently, while rental customers are subject to local and federal regulations related to the treatments, the rental fee for the devices may be low.

The terms renting and leasing are used interchangeably throughout. These terms also encompass purchasing a license to use the device.

There is provided in accordance with a non-limiting embodiment of the invention a system for treatment including an identification (ID) element, an ID reader configured to read ID information provided by the ID element, the ID information including an identifier of a user and authorization information for authorizing that user to use the medical device, the authorization information including authorization that the user has paid a rental fee to use the medical device, a controller in communication with the ID reader configured to process the ID information, and a disabling element capable of disabling operation of the medical device upon receiving a disabling signal from the controller if the controller cannot verify that the user is authorized to use the medical device.

In accordance with a non-limiting embodiment of the invention the ID element is wearable by the user. In accordance with a non-limiting embodiment of the invention the ID element is mountable on the medical device.

In accordance with a non-limiting embodiment of the invention the disabling element is mountable in or on the medical device.

In accordance with a non-limiting embodiment of the invention the medical device includes an energy-emitting medical device.

In accordance with a non-limiting embodiment of the invention the energy-emitting medical device includes a low-intensity shockwaves device.

In accordance with a non-limiting embodiment of the invention the energy-emitting medical device includes a sexual wellness improvement device.

In accordance with a non-limiting embodiment of the invention the energy-emitting medical device includes a device for treatment of erectile dysfunction.

In accordance with a non-limiting embodiment of the invention the energy-emitting medical device includes a device for treatment of a diabetic ulcer, a vagina, pain, or cosmetic treatment.

In accordance with a non-limiting embodiment of the invention the authorization information includes authorization that the user has passed a training course as a prerequisite for renting the medical device.

In accordance with a non-limiting embodiment of the invention the authorization information includes detection if the medical device is located in an authorized treatment area.

In accordance with a non-limiting embodiment of the invention the authorization information includes authorization that the user is under medical supervision while in an authorized treatment area during use of the medical device.

### BRIEF DESCRIPTION OF THE DRAWINGS

The invention may be more clearly understood upon reading of the following detailed description of non-limiting exemplary embodiments thereof, with reference to the following drawings, in which:

FIG. 1 is a block diagram of rental medical devices, in accordance with embodiments of the present invention.

FIG. 2 is a block diagram of system for using rental medical devices, in accordance with embodiments of the present invention.

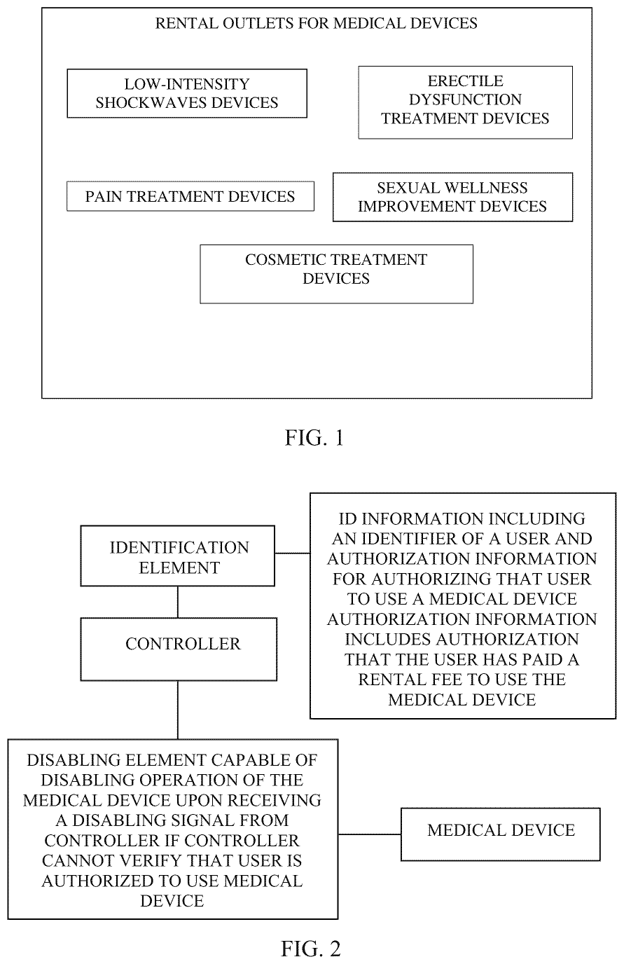

### DETAILED DESCRIPTION

The present invention seeks to provide medical device rental outlets (as opposed to medical clinics) for renting to users on a per-sessions basis electromedical devices intended for multi-sessions treatments of various indications at the outlet or close to it. As opposed to medical clinics, such outlets are merely rental outlets, and as such, are subject to less regulations, are easier to manage, and are less expensive to set up and run.

Presently, there are medical devices that are rented to users of home healthcare equipment, such as beds, wheelchairs, inhalators, electrical muscle stimulators, and many more. Other energy-emitting electromedical devices configured and regulated for home-use may be used for treatment of pain reduction, erectile dysfunction, diabetic foot ulcers, and many more. Such energy-emitting treatments, called sequential treatments, may include multiple sessions of less than one hour each, with inter-session breaks of several days each.

Sequential treatments performed as home treatments have several disadvantages. For the lessee (home user) of the equipment, renting or leasing a device for the treatment duration can be very expensive due to the relatively long time duration of the treatment. In other words, although each session may be short, the whole treatment extends over a long period of time. Thus, the rental/leasing fee for the duration of the treatment is prohibitively high.

For the lessor (owner or licensee) of the equipment, sequential treatments performed as home treatments have a low rental throughput (low number of treatments per unit time), and there is a relatively high and expensive risk of device damage during treatment and transportation.

Alternatively, setting up a mobile operation, in which the medical devices may be moved from patient to patient, may increase the throughput of home treatments per device, but the rental cost increases due to the device transportation, regulatory requirements for performing treatment and added risk of damage. Moreover, operating several devices in parallel requires an operator for each device.

Thus, a low cost high throughput system is desired for sequential treatments.

The invention solves these problems by providing a network system for affordable rental of medical devices configured for home use. Since home-use devices can also be used outside the user's home, the invention provides a leasing/rental network of outlets for renting such home-use devices to be self-operated by users (as opposed to by medical staff) at or geographically close to the outlet. Since multiple users may perform sessions of sequential self-treatments in a common site using a common device, a high throughput and low operational cost may be achieved, in particular when a single trained operator simultaneously assists several self-treating users in operating respective devices.

Accordingly, each treatment is composed of sequentially-performed multiple sessions, which take place at a common site. The medical devices used in the system of the invention, include, without limitation, energy-emitting devices configured to deliver treating energy. In one aspect of the invention, no person other than the patient needs to contact the device during the delivery. Alternatively, the patient may be assisted, when needed, by one or more trained operators at the medical-device rental outlet. The devices used may be devices which have been configured for home treatment.

In one non-limiting example, the invention provides a site for users to rent energy-emitting devices for sequentially-performed multiple sessions for indications such as but not limited to, sexual wellness improvement (e.g., treatment of erectile dysfunction), or diabetic foot ulcer, and many others. One aspect of the invention is providing sequentially-performed multiple sessions using a low-intensity shockwaves device, which may be used for treating the area of the pelvis, peritoneum or penis, e.g., for treatment of erectile dysfunction, or other tissues and parts of the body, such as but not limited to, vaginal treatments, pain and cosmetic indications, and others.

The invention provides outlets that take advantage of the short sessions and alleviate the cost of long treatment by offering usage of an available device in an outlet for the duration of the session; there is no renting a particular device but sharing usage of available devices for self-treatments.

Sharing sites and devices increase devices usability and may reduce user expense for such a treatment. In addition, sessions can take place in various outlets, so there is no need to transport a device from place to place, in case of the patient traveling.

Thus, in one aspect of the invention, the outlets provide shared usage of devices by more than one self-treating patient where the treatments include multiple sessions that are performed over a period of time.

Some non-limiting features of the invention include requiring a user to pass a training course as a prerequisite for rental transactions. Another non-limiting feature is requiring or allowing the user to rent a combination of a device and a treatment area for using the device. The treatment area may be a closed room or a portion of a room, or an outdoor area, such as a fenced-in area designated for treatment. Another non-limiting feature is not allowing the user to transport the device. Another non-limiting feature is requiring the user to be under medical supervision while in the rented treatment area throughout the period of using the device.

The invention provides devices that ensure these features are carried out. For example, some non-limiting features of the invention include requiring a user to pass a training course as a prerequisite for rental transactions. Another non-limiting feature is requiring or allowing the user to rent a combination of a device and a treatment area for using the device. The treatment area may be a closed room or a portion of a room, or an outdoor area, such as a fenced-in area designated for treatment. Another non-limiting feature is not allowing the user to transport the device. Another non-limiting feature is requiring the user to be under medical supervision while in the rented treatment area throughout the period of using the device.

What is claimed is:

1. A system for treatment comprising:
an identification (ID) element;
an ID reader configured to read ID information provided by said ID element, said ID information comprising an identifier of a user and authorization information for authorizing that user to use said medical device, said authorization information comprising authorization that said user has paid a rental fee to use said medical device;
a controller in communication with said ID reader configured to process said ID information; and
a disabling element capable of disabling operation of said medical device upon receiving a disabling signal from said controller if said controller cannot verify that said user is authorized to use said medical device.

2. The system according to claim 1, wherein said ID element is wearable by said user.

3. The system according to claim 1, wherein said ID element is mountable on said medical device.

4. The system according to claim 1, wherein said disabling element is mountable in or on said medical device.

5. The system according to claim 1, wherein said medical device comprises an energy-emitting medical device.

6. The system according to claim 5, wherein said energy-emitting medical device comprises a low-intensity shockwaves device.

7. The system according to claim 5, wherein said energy-emitting medical device comprises a sexual wellness improvement device.

8. The system according to claim 5, wherein said energy-emitting medical device comprises a device for treatment of erectile dysfunction.

9. The system according to claim 5, wherein said energy-emitting medical device comprises a device for treatment of a diabetic ulcer.

10. The system according to claim 5, wherein said energy-emitting medical device comprises a device for treatment of a vagina.

11. The system according to claim 5, wherein said energy-emitting medical device comprises a device for treatment of pain.

12. The system according to claim 5, wherein said energy-emitting medical device comprises a device for cosmetic treatment.

13. The system according to claim 1, wherein said authorization information comprises authorization that said user has passed a training course as a prerequisite for renting said medical device.

14. The system according to claim 1, wherein said authorization information comprises detection if said medical device is located in an authorized treatment area.

15. The system according to claim 1, wherein said authorization information comprises authorization that said user is under medical supervision while in an authorized treatment area during use of said medical device.
